(19) United States
(12) Patent Application Publication
(10) Pub. No.: US 2009/0246881 A1
(43) Pub. Date: Oct. 1, 2009

(54) Detection of Explosives Using Luminescence

(75) Inventors: Sarah Josepha Toal, Rockville, MD (US); Jennifer Marie Sievering, Gaithersburg, MD (US)

Correspondence Address: DIEDERIKS & WHITELAW, PLC, 12471 DILLINGHAM SQUARE, #301, WOODBRIDGE, VA, 22192, US

(73) Assignee: REDXDEFENSE, LLC, Rockville, MD (US)

(21) Appl. No.: 12/442,160

(22) PCT Filed: Sep. 21, 2007
(86) PCT No.: PCT/US07/20442

§ 371 Date: Mar. 20, 2009

Related U.S. Application Data
(60) Provisional application No. 60/846,496, filed on Sep. 22, 2006.

(51) Int. Cl. G01N 33/22; G01N 21/75

(52) U.S. Cl. 436/110; 422/82.08; 436/106

(57) ABSTRACT

A system and method for detecting trace explosives is provided that utilizes a lumnescent reagent, such as blue-fluorescent biphenyl compound, to simultaneously detect multiple nitrogen-based explosives on a sample substrate. The test reagent is mixed with a solvent to improve mixing and maximize the dissolution of the reagent and any trace explosives present on the substrate. A thin film of reagent is applied to the substrate either before or after the substrate contacts a sample area to be tested. Heated air is then applied to the substrate to improve the sample reading. A light source is utilized to illuminate the reagent and expose any quenched portions of the substrate that indicate the presence of an explosive. Quenched portions may be detected visually, or utilizing an analyzing apparatus, such as a fluorimeter or camera. A computer may also be utilized to interpret and record test results.

### CROSS-REFERENCE TO RELATED APPLICATIONS

The present application represents a National Stage application of PCT/US2007/020442 filed Sep. 21, 2007 entitled “Detection of Explosives Using Luminescence”, and further claims the benefit of U.S. Provisional Patent Application Ser. No. 60/846,496 entitled “Detection of Explosives using Luminescence and Chemiluminescence” filed Sep. 22, 2006.

### BACKGROUND OF THE INVENTION

1. Field of the Invention

The present invention pertains to the art of explosive detection and, more particularly, to the use of luminescent compounds for the detection of trace explosives.

2. Discussion of the Prior Art

The detection of small amounts of explosives is significantly important for the prevention of terrorist attacks and for the safeguarding of military personnel and bases, airports and other transportation locations, and tourist and commercial venues. Chemical sensors are often desired because they are able to detect trace amounts of explosives and can be packaged into simple-to-use, low-cost devices. In contrast, conventional detection methods, such as X-ray diffraction, nuclear quadrupole resonance, and gas chromatography-mass spectrometry, though highly sensitive, are expensive, difficult to maintain, susceptible to false-positives, and are not easily manufactured into low-power, portable devices.

The low volatility of many explosives, such as trinitrotoluene (TNT) and particularly cyclo trimethylene trinitramine (RDX) and pentaerythritol tetranitrate (PETN), makes vapor sampling difficult and largely impractical. Thus, efficient solid-state sampling techniques are desirable for many applications. Explosive residue, in the form of particulates, is known to contaminate persons, hair, skin, clothing, work-surfaces, floors and other materials during the preparation and packaging of explosive devices. This widespread contamination presents significant opportunity to spread contamination to surfaces outside explosives and bomb-making factories through the transport of contaminated products in cars and from the direct contact of contaminated persons or objects with other materials. Portable calorimetric chemical sensing kits have the value of displaying easily interpreted optical signals with fast response times. However, there is currently a demand for explosives sensors that maintain the simplicity of use found in calorimetric kits, but with improved detection sensitivities and lower cost per sample.

### SUMMARY OF THE INVENTION

A system and method for detecting trace explosives is provided that utilizes a lumnescent reagent, such as blue-fluorescent biphenyl compound, to detect trace explosives on a sample substrate. A preferred embodiment of the present invention is concerned with a method for the simultaneous detection of one or more nitrogen-based explosives, including nitroaromatic-, nitramine- and organic nitrate-based explosives, which may be present on a sampling substrate, using a blue-fluorescent biphenyl test reagent. The test reagent is mixed with a solvent to improve mixing and maximize the dissolution of the reagent and any trace explosives present on the substrate. Preferably, and inorganic solvent is utilized, such as acetone, toluene, tetrahydrofuran (THF), or a combination thereof. A thin film of reagent is applied to the substrate either before or after the substrate is placed into contact or swiped over a sample area to be tested. Alternatively, the sample substrate may be the area to be tested itself. In a preferred embodiment, reagent is applied utilizing an aerosol container. Forced heated air is optionally applied to the substrate to speed the evaporation of solvent, so that observation of luminescence may be performed more rapidly. Heat further serves to increase the rate of interaction between any explosives particulates and the reagent, and may help partially vaporize the explosive residue, which improves diffusion and condensation of the residue into the sensing material or sample substrate. A light source, such as a UV lamp, UV LED's, or UV cathode tubes, is utilized to illuminate the reagent. When present, explosives will provide a quenching effect to the luminescent sample substrate, allowing for easy visual detection of trace contaminates. Alternatively, quenched portions may be detected utilizing a detection apparatus, such as a fluorimeter or camera. A computer may also be utilized to interpret and record test results.

A versatile system and method is therefore provided which allows for one-swipe simultaneous detection of multiple explosives, resulting in a quick, accurate and inexpensive test procedure. Additional objects, features and advantages of the present invention will become more readily apparent from the following detailed description of preferred embodiments when taken in conjunction with the drawings wherein like reference numerals refer to corresponding parts in the several views.

### BRIEF DESCRIPTION OF THE DRAWINGS

FIG. 1 is a perspective view of a screening system employed in connection with the invention;

FIG. 2 is a cut-away side view of the screening system of FIG. 1;

FIG. 3 is a front right perspective view of a fully integrated portable screening system employed in connection with the present invention;

FIG. 4 is a rear perspective view of the screening system of FIG. 3;

FIG. 5 is a left side, cut-away view of the portable screening system of FIGS. 3 and 4;

FIG. 6 is an exemplary image of quenching of the luminescence of a thin film of a blue-fluorescent biphenyl compound by the explosives (from top to bottom) TNT, RDX, PETN, and Tetryl, which have been deposited from acetonitrile solutions. Seven contamination spots, decreasing from left to right, have been deposited which span over a 10-fold range in concentration; an acetonitrile blank spot was placed immediately to the right of the seventh explosive contaminated spot (no quenching observed).

### DETAILED DESCRIPTION OF THE INVENTION

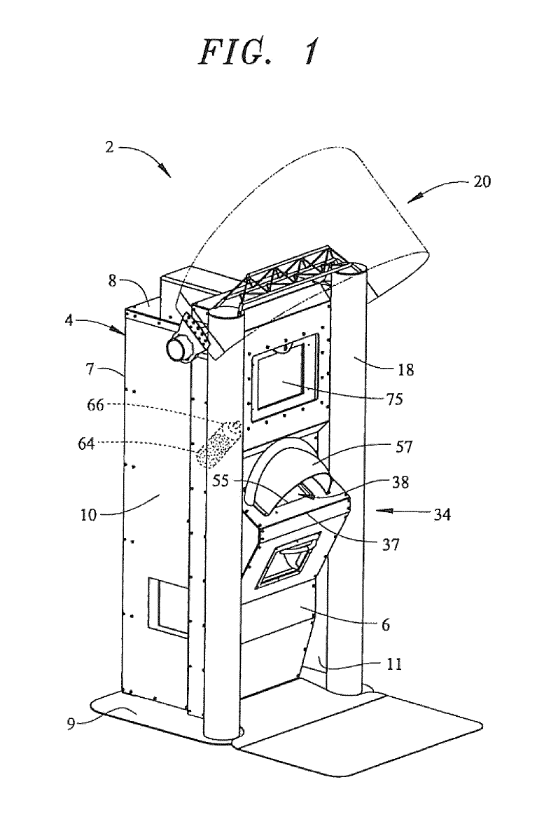

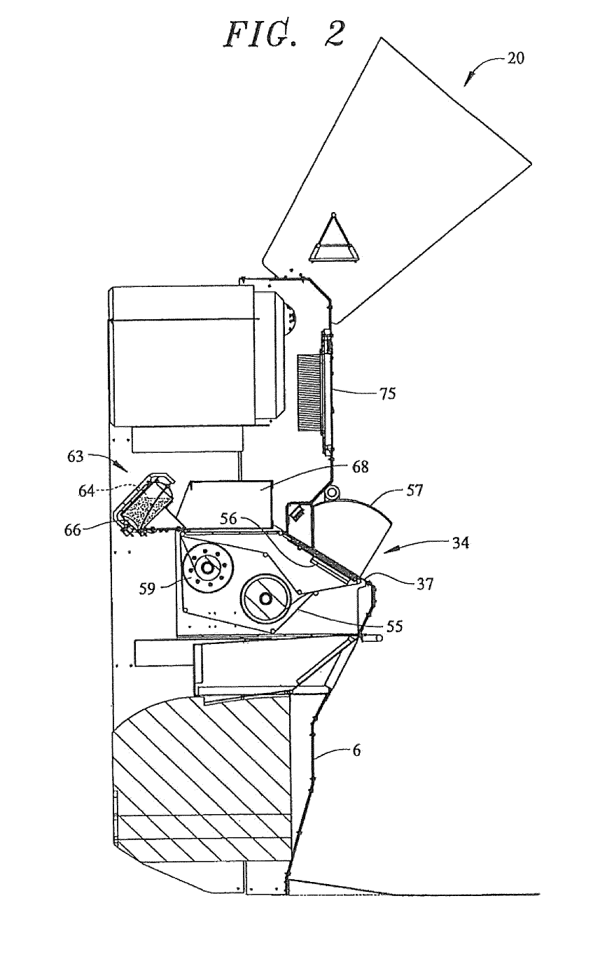

With initial reference to FIGS. 1 and 2, a personnel screening system or kiosk 2 for the detection of explosives which can utilize in connection with the present invention. Screening system 2 includes a main housing 4 provided with a front wall 6, a rear wall 7, a top wall 8, a bottom wall or base 9 and opposing side walls 10 and 11. In addition, screening system 2 is provided with various accessories. For example, in the embodiment shown, screening system 2 is shown with a pair of columns 17 and 18, as well as a shield 20.

In any event, screening system 2 includes a collecting portion 34, arranged within a housing 37, that retrieves trace residue samples and certain demographic samples from a subject. As such, collecting portion 34 includes a residue sample collector 38 having a sample collecting sheet or substrate 55 positioned upon a palm pad 56 below a cowl 57. Sample collecting sheet 55 is provided on a continuous roll 59 that enables screening system 2 to provide a clean sheet for each subject. In any case, sample collecting sheet 55 includes a slightly adhesive or tacky surface that attracts trace residue from the subject. In addition to obtaining trace residue, the sample collecting sheet 55, in combination with palm pad 56, can simultaneously obtain a biometric indicator, such as a handprint or fingerprint, in a manner that will be discussed more fully below. Once a trace residue sample has been collected from the subject, sample collecting sheet 55 is moved to a sample analyzer 63. Upon reaching sample analyzer 63, a test reagent 64 stored within a test reagent dispenser 66 is sprayed onto sample collecting sheet 55 and a test for a threat residue by an analyzing portion 68, such as a camera, fluorimeter or spectrometer, of sample analyzer 63 is performed, while results can be presented on a display 75.

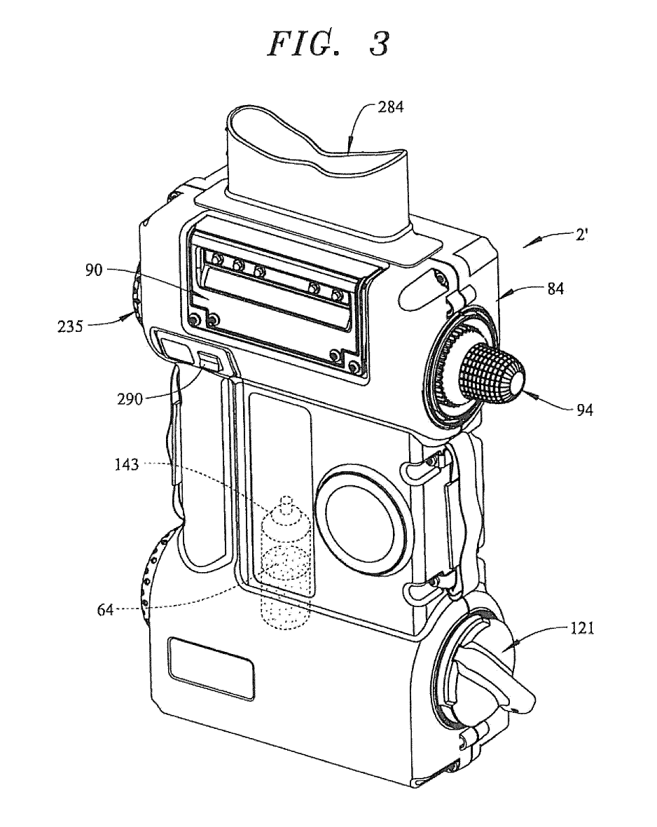

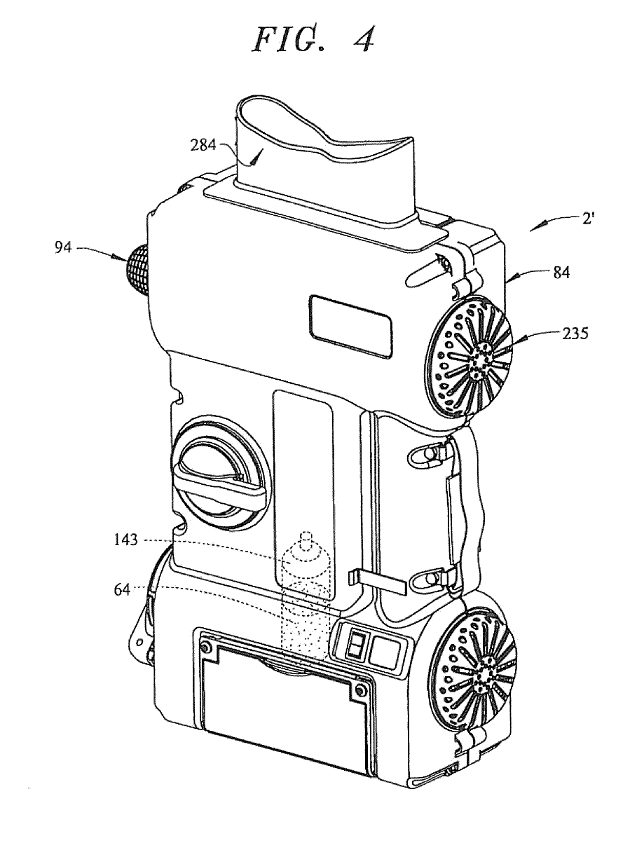

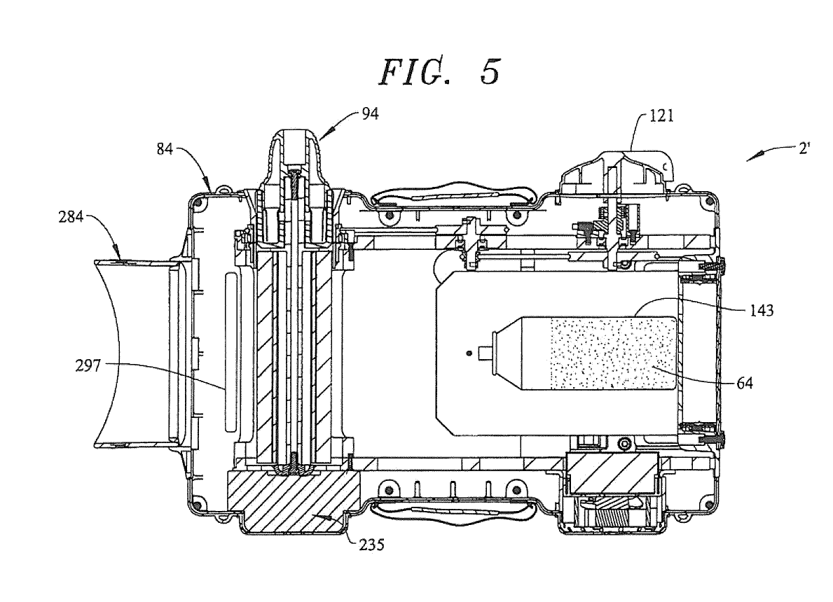

The present invention is particularly concerned with detecting nitroaromatic-, nitramine- and organic nitrate-based explosives through the use of a single test reagent containing a luminescent compound as will be detailed more fully below. However, at this point, it should be understood that screening system 2 can take various forms within the scope of the invention. For instance, although screening system 2 generally represents a fixed screening arrangement, the invention can be equally employed with a portable screening or detection system. For instance, FIGS. 3-5 illustrate an embodiment of a self contained, fully integrated, portable screening system 2′ for determining whether a subject has been in contact with a particular analyte of interest. By portable, it should be understood that the present invention is a small (no larger than a briefcase) light unit that is readily transportable and deployable by a single individual operator. Screening system 2′ includes a housing 84 having an indicator panel 90 provided on a front wall (not labeled) thereof. Portable screening system 2′ employs a contact pad or baton 94 to obtain a trace sample which potentially contains an analyte of interest from a subject. To obtain a sample, an individual is asked to grasp baton 94 after baton 94 has been removed from housing 84. Alternatively, baton 94 can be rolled or brushed across an object to obtain a trace sample.

Baton 94 includes a plurality of individual single collection sheets (not labeled). After each screening, the used sample collection sheet is removed to expose a new, pristine sample collection sheet for a subsequent testing procedures. Each sample collection sheet is preferably coated with a tacky adhesive that retains trace residue and any potential analytes of interest obtained from the subject. After obtaining a trace sample from a test subject, the sample collection sheet is exposed to an luminescent test medium or reagent 64 which, in accordance with the invention as indicated above, is capable of simultaneously detecting nitroaromatic-, nitramine- and organic nitrate-based explosives through the use of a single test reagent. As shown, test reagent 64 is housed in a solution container 143 located within the outer container or housing 84 of the portable device 2′ and is adapted to be delivered by rotation of a manual pump unit 121.

Following the collection of a sample to be tested and the application of the test reagent 64, a heated forced air supply is provided by a convection dryer unit 235 which is preferably activated to dry sample collection sheet or baton 94. Therefore, dryer unit 235 preferably employs a combination heater and fan to dry the sample collection sheet. After the sample collection sheet is dry, the sample is ready for viewing. At this point, the operator peers through a view finder 284 and presses view switch 290 to activate a testing mechanism, preferably in the form of a light source 297 and most preferably in the form of an ultraviolet light source, which will excite the luminescent compound of the test reagent 64 in order to enable the simultaneous visual detection of any of the earmarked explosives.

In any case, as indicated above, the particular form of test reagent application and sample analysis can vary without departing from the invention. The invention is particularly suitable for use in connection with the screening systems disclosed in U.S. patent application Ser. Nos. 11/418,144, 11/418,193 and 11/525,509, which provide greater details of many features of the screening systems discussed above and are incorporated herein by reference. However, it should be understood that the invention can even be carried out by directly applying the luminescent test reagent directly to a sample substrate, such as by providing the test reagent in a portable aerosol container. Advantageously, a single, particular luminescent test reagent is provided for simultaneously detecting a wide range of nitrogen-based explosives, including nitroaromatic-, nitramine- and organic nitrate-based explosives such that the need for separate tests, with distinct reagents, for each type of explosive can be avoided as detailed further below.

The present invention particularly concerns employing a fluorimetric method with a substantially universal, luminescent test reagent for detecting trace quantities of nitrogen-based explosive residue, including nitroaromatic-, nitramine- and organic nitrate-based explosives. This invention allows for the detection of a wide range of explosive classes in one step, thereby offering a means for simple, highly sensitive, and low-cost explosive sensors. Additionally, various embodiments allow for sampling from a wide range of surfaces, including hands, clothing, cars, packages, door handles, buildings, desks, computers, and more.

Many nitrogen-based explosives are electron-acceptors. In nitroaromatics, for example, the π* lowest unoccupied molecular orbitals (LUMOs) are of low energy due to the electron-withdrawing effect of the nitro substituent on the aromatic ring. A higher degree of nitro-substitution results in a higher reduction potential and a greater oxidizing ability (nitrobenzene (−1.15 V), dinitrotoluene (−0.9 V), and trinitrotoluene (−0.7 V), versus normal hydrogen electrode (NHE)). Similarly, organic molecules functionalized with nitro groups have lower energy LUMOs, which increase their oxidizing abilities. Thus, organic nitro compounds, such as the nitramine explosives (e.g. RDX) and the organic nitrates (e.g. PETN and nitroglycerin), have increased electron-accepting abilities compared to many other organic compounds.

Luminescent compounds are often highly conjugated chemicals and are found in a variety of chemical classes, including aromatic hydrocarbons such as 9,10-diphenylanthracene, aromatic heterocycles such as 2,3-diaminonapthalene, conjugated organic polymers such as polyfluorenes, and inorganic polymers such as polymetalloles, to name a few. Conjugation of electrons results in a low energy π* lowest unoccupied molecular orbital and a low energy delocalized excited state. Delocalization of the excited state in conjugated polymers is advantageous in that exciton migration increases the frequency of quenching events with bound analytes. Conjugated compounds are electron donors. Thus, these luminescent materials may be used for redox sensing of electron-deficient analytes, such as nitrogen-based explosives, through electron-transfer luminescence quenching.

The present invention employs the use of luminescent compounds to effect the detection of nitrogen-based explosives through luminescence quenching. Direct interaction of an electron-accepting analyte to a luminescent polymer can cause luminescence quenching, which may be monitored to identify the presence of explosives on a sampling substrate. In one exemplary method, a sampling substrate is first exposed to an environment suspected of being contaminated with explosives, subsequently exposed to a test reagent containing a luminescent compound, then observed to determine the presence of explosives through luminescence quenching. Luminescent compounds may be monomers, oligomers, polymers or copolymers. More Specifically, luminescent compounds which may be utilized with the present invention include luminescent polyacetylenes, polyvinylenes, polyphenylenes, polyfluorenes, poly(p-phenyleneethynylenes), poly(p-phenylenevinylenes), poly(fluorenylvinylenes), poly(fluorenylethynylenes), poly(fluoreneylphenylenes), polycarbazoles, carbazole-containing polymers, or biphenyls. In a preferred embodiment, the luminescent compound is a blue-flourescent biphenyl compound 3,3′-[(9,9-dihexyl-9H-fluorene-2,7-diyl)-di-2,1-ethenediyl]bis[9-ethyl-9H-carbazole]. One exemplary sampling substrate is filter paper that may be contacted to an external surface. However, other suitable sampling substrates may be utilized such as cellulose-based paper, glass fiber paper or chromatography paper. Regardless, the sample substrate should be chosen such that the luminescence from the sample substrate minimally interferes with the luminescence from the test reagent. In a preferred embodiment, the sampling substrate of choice includes an adhesive thereon to aid in the collection of a sample. While the sampling substrate may be a material or surface that is exposed to an environment suspected of being contaminated with explosives, the sampling substrate may also be the suspected contaminated surface itself.

Once the sampling substrate is prepared, it is exposed to a detection reagent that contains a luminescent compound. In one preferred embodiment, the detection reagent contains a luminescent compound dissolved in a solvent. One or more solvents are preferably selected to maximize dissolution of the luminescent compound and any trace residue, such as explosives, present in the sample and to promote mixing thereof. Solvents contemplated for use with the present invention include alcohols, diethyl ether, pentane(s), hexane(s), xylene(s), water, ethyl acetate, acetonitrile, dimethyl sulfoxide (DMSO), N-Methyl-2-pyrrolidone (NMP) and dimethylformamide (DMF). Preferably, a volatile organic solvent is utilized such as acetone, toluene, or combinations thereof. In addition, the luminescent compound is preferably chemically modified to increase its solubility in preferred solvents, or chosen in part by reference to its solubility.

The reagent may be applied to the sampling substrate by various means. A test reagent container can be any suitable container, such as a metal pressure vessel, a microcapsule or a plastic container. In a preferred embodiment, the reagent is packaged into an aerosol can. The reagent is then applied to the sampling substrate by spraying the reagent onto the substrate, creating a thin film of the luminescent material on the substrate. The sampling substrate is then put into a dark environment and exposed to a wavelength of light capable of exciting luminescence from the reagent. The excitation source utilized with the present invention may be a black light, a blue light, a white light, a mercury-deuterium lamp, xenon-arc lamp, light emitting diodes, or cathode ray tubes, for example. The excitation source or light source should be chosen to maximize excitation of the luminescent compound while simultaneously minimizing the degree of photodegredation. Preferably, the excitation source is a UV lamp, UV LED's or UV cathode tubes. Detection is confirmed by noticing dark quenched spots amidst the bright background of the luminescent test reagent. Preferably, the thickness of the thin film is controlled so that the signal-to-noise ratio of the quenching of luminescence will be optimized. A film that is too thick may create an excess of luminescence such that quenching will not be observed. A film that is too thin may inhibit adequate detection due to low emission intensity resulting in a low signal-to-noise ratio. In other embodiments, the reagent is applied using a pressurized vessel or pumping mechanism, an airbrush, an electrically powered constant pressure cylinder or by drop-coating the reagent onto the substrate to create a thin film of the luminescent material. Additionally, the application system can include solenoid gate nozzles connected to the reagent container or a mechanical press. Alternatively, application of the reagent may include the manual rupturing of microcapsules of reagent. The sampling substrate may also be submerged into the reagent. It should be understood that the type of packaging utilized for the test reagent depends on the particular detection system utilized. Regardless, the packaging is preferably designed to optimize delivery of the test reagent to the sample substrate. In another embodiment, the reagent is applied to the sampling substrate prior to exposure to the surface suspected of being contaminated with explosives. The reagent may also be embedded into the sampling substrate prior to surface sampling.

In one preferred embodiment of the product, the sampling substrate is exposed to heat and/or forced air flow after the contact of the sampling substrate and the surface suspected of being contaminated by explosives. This serves multiple purposes, one of which is to speed the evaporation of solvent, so that observation of the luminescence and luminescence quenching may be performed more rapidly. Heat further serves to increase the rate of interaction between the explosives particulates and the luminescent sensing material. Heat may help partially vaporize the explosive residue, which will improve its ability to diffuse into, and subsequently condense into the sensing substrate. Quenching is then observed once there is sufficient mixing of explosive analyte and sensor material or reagent to effect quenching. If a compound is luminescent in the visible spectrum, the quenching may be observed either through direct visual examination or indirect visual examination using a camera or other instrumentation as an intermediary. In addition, the quenching may be recorded with the use of a visible or ultra-violet camera, or with the use of a fluorimeter or fluorescence spectrometer. The instrumentally recorded data may be analyzed directly or by using computer software to interpret results and make a determination of whether or not explosives are present.

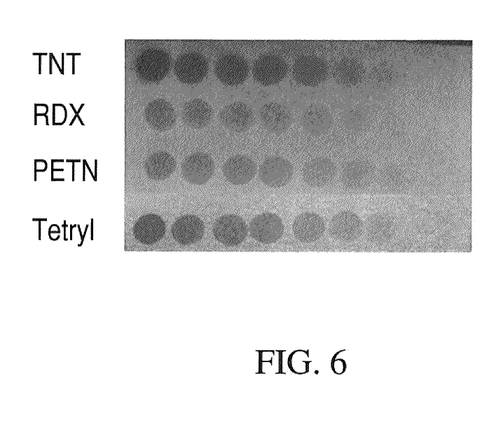

Your attention is drawn to FIG. 6, which is an exemplary image of quenching of the luminescence of a thin film of a blue-fluorescent biphenyl compound by the explosives (from top to bottom) TNT, RDX, PETN, and Tetryl, which have been deposited from acetonitrile solutions. Seven contamination spots, decreasing from left to right, have been deposited which span over a 10-fold range in concentration; an acetonitrile blank spot was placed immediately to the right of the seventh explosive contaminated spot (no quenching observed).

As should be evident from the discussion above, the present invention provides improved detection sensitivities in an inexpensive and easy to use system. Advantageously, multiple nitrogen-based explosives can be detected simultaneously during a single reading utilizing minimal materials. Thus, trace amounts of explosives that would otherwise be overlooked when utilizing previous multi-stage/reagent systems, may be detected with a single swipe of a sample substrate over a surface of interest. Although described with reference to preferred embodiment of the invention, it should be readily understood that various changes and/or modifications can be made to the invention without departing from the spirit thereof. In general, the invention is only intended to be limited by the scope of the following claims.

We claim:

1. A system for the detection of explosives comprising:
a sample substrate for collecting a sample which may include trace explosives;
a container housing a test reagent, said test reagent containing a luminescent compound which, upon excitation luminesces and whose luminescence is capable of being quenched by the presence of any one or more of nitroaromatic-, nitramine- and organic nitrate-based explosives;
an application system for selectively directing the test reagent onto the sample substrate; and
a testing device for exciting the test reagent and enabling the simultaneous detection of any one or more trace nitroaromatic-, nitramine-, organic nitrate-based explosives in the sample based on a presence or absence of luminescence.

2. The system of claim 1, wherein said luminescent compound is a luminescent monomer, oligomer, polymer, or copolymer.

3. The system of claim 2, wherein the luminescent compound is a substance 3,3′-[(9,9-dihexyl-9H-fluorene-2,7-diyl)di-2,1-ethenediyl]bis[9-ethyl-9H-carbazole].

4. The system of claim 2, wherein said luminescent compound is selected from the group consisting of luminescent polyacetylenes, polyvinylenes, polyphenylenes, polyfluorenes, poly(p-phenyleneethynylenes), poly(p-phenylenevinylenes), poly(fluorenylvinylenes), poly(fluorenylethynylenes), poly(fluoreneylphenylenes), polycarbazoles, carbazole-containing polymers, and biphenyls.

5. The system of claim 1, wherein said test reagent contains one or more solvents selected to maximize dissolution of the luminescent compound and any trace explosives present in the sample and promote efficient mixing thereof.

6. The system of claim 5, wherein said solvents are selected from the group consisting of alcohols, diethyl ether, tetrahydrofuran, acetone, pentane(s), hexane(s), toluene, xylene(s), water, ethyl acetate, acetonitrile, dimethyl sulfoxide, N-Methyl-2-pyrrolidone, dimethylformamide and mixtures thereof.

7. The system of claim 1, further comprising a heater for heating the sampling substrate following application of the test reagent to the sample substrate.

8. The system of claim 1, further comprising a forced air supplier for introducing forced air flow across the sample substrate to speed the evaporation of solvent within said test reagent.

9. The system of claim 1, further comprising a casing within which the sample substrate and testing device are located, said casing also including a visual inspection port for observing the presence or absence of luminescence.

10. The system of claim 1, wherein said step of observing the absence of luminescence involves using a fluorimeter or a camera.

11. The system of claim 9, further comprising a computer for recording data and interpreting results.

12. The system of claim 1, wherein said sample substrate is selected from the group consisting of filter paper, cellulose-based paper, glass fiber paper and chromatography paper.

13. The system of claim 1, wherein said container could be a structure selected from the group consisting of an aerosol can, a metal pressure vessel, a microcapsule and a plastic container.

14. The system of claim 1, wherein said application system is selected from the group consisting of solenoid gated nozzles connected to said container, a mechanical press, and the act of applying pressure to rupture microcapsules.

15. The system of claim 1, wherein said application system results in a test reagent being sprayed onto a sample substrate, the spray being driven by one of the following: propellant(s) within an aerosol can, a pressurized vessel, an airbrush or an electrically powered constant pressure cylinder.

16. The system of claim 1, wherein said sample substrate is an environment suspected of being contaminated with explosives itself.

17. The system of claim 1, where said testing device is an excitation source selected from the group consisting of a black light, a blue light, a white light, a mercury-deuterium lamp, xenon-arc lamp, light emitting diodes, and cathode ray tubes.

18. The system of claim 1, wherein said sample substrate is provided with an adhesive.

19. The system of claim 1, wherein said sample substrate contains the test reagent therein or thereon prior to collecting the sample.

20. A method of detecting explosives comprising:
collecting a sample, which may contain traces of any one or more of nitroaromatic-, nitramine-, and nitrate ester-based explosives, onto a sample substrate;
applying a test reagent onto the sample substrate, wherein the test reagent contains a luminescent compound which, upon excitation luminesces and whose luminescence is capable of being quenched in the presence of any one or more of nitroaromatic-, nitramine- and organic nitrate-based explosives;
exciting the test reagent; and
simultaneously determining whether traces of any one or more of nitroaromatic-, nitramine- and organic nitrate-based explosives are present on the sample substrate based on a presence or absence of luminescence.

21. The method of claim 20, wherein determining the presence or absence of luminescence is performed through visual inspection.

22. The method of claim 20, wherein determining the presence or absence luminescence includes the use of a fluorimeter or camera.

23. The method of claim 21, wherein said step of determining the presence of absence of luminescence is performed by a computer.

24. The method of claim 20, further comprising spraying the test reagent onto the sample substrate to create a thin film of the luminescent compound.

25. The method of claim 20, further comprising selecting a sample substrate such that the luminescence from said sample substrate minimally interferes with the luminescence from the test reagent.

26. The method of claim 20, further comprising selecting a light source or sources to maximize excitation of the luminescent compound while simultaneously minimizing degree of photodegradation.

27. The method of claim 20, further comprising mixing the test reagent with a solvent mixture designed to maximize dissolution of the luminescent compound and any trace explosives present in the sample and promote efficient mixing thereof.

28. The method of claim 20, further comprising packaging the test reagent in manner designed to optimize delivery of the test reagent to said sample substrate.

29. The method of claim 20, wherein said sample substrate contains the test reagent therein or thereon prior to collecting the sample.

30. The method of claim 20, wherein said test reagent is contained in microcapsules.

31. The method of claim 20, further comprising heating the sample substrate following exposure to the test reagent to increase mixing of the test reagent and explosives or to speed solvent evaporation.

32. The method of claim 20, further comprising introducing forced air flow across the sample substrate to speed the evaporation of solvent within said test reagent.

33. The method of claim 20, wherein said luminescent compound is a luminescent monomer, oligomer, polymer, or copolymer.

34. The method of claim 33, further comprising employing 3,3′-[(9,9-dihexyl-9H-fluorene-2,7-diyl)di-2,1-ethenediyl]bis[9-ethyl-9H-carbazole] as the luminescent compound.

35. The method of claim 33, wherein said luminescent compound is selected from the group consisting of luminescent polyacetylenes, polyvinylenes, polyphenylenes, polyfluorenes, poly(p-phenyleneethynylenes), poly(p-phenylenevinylenes), poly(fluorenylvinylenes), poly(fluorenylethynylenes), poly(fluoreneylphenylenes), polycarbazoles, carbazole-containing polymers, and biphenyls.

36. The method of claim 20, wherein said sample substrate is an environment suspected of being contaminated with explosives itself.
